(19) United States
(12) Patent Application Publication
(10) Pub. No.: US 2026/0327167 A1
(43) Pub. Date: Oct. 22, 2026

(54) PREPARATION METHOD AND PRODUCTION SYSTEM FOR HEXAFLUOROBUTADIENE

(71) Applicants: SINOCHEM LANTIAN ELECTRONIC MATERIALS (HANGZHOU) CO., LTD., Hangzhou, Zhejiang (CN); ZHEJIANG RESEARCH INSTITUTE OF CHEMICAL INDUSTRY CO., LTD, Hangzhou, Zhejiang (CN); SINOCHEM LANTIAN CO., LTD., Hangzhou, Zhejiang (CN)

(72) Inventors: Haifeng WU, Hangzhou, Zhejiang (CN); Wucan LIU, Hangzhou, Zhejiang (CN); Hao CHENG, Hangzhou, Zhejiang (CN); Yongying LIU, Hangzhou, Zhejiang (CN); Zhongjie SHU, Hangzhou, Zhejiang (CN)

(73) Assignees: SINOCHEM LANTIAN ELECTRONIC MATERIALS (HANGZHOU) CO., LTD., Hangzhou, Zhejiang (CN); ZHEJIANG RESEARCH INSTITUTE OF CHEMICAL INDUSTRY CO., LTD, Hangzhou, Zhejiang (CN); SINOCHEM LANTIAN CO., LTD., Hangzhou, Zhejiang (CN)

(21) Appl. No.: 18/877,726

(22) PCT Filed: Nov. 15, 2024
(86) PCT No.: PCT/CN2024/132457

§ 371 Date: Dec. 20, 2024

(30) Foreign Application Priority Data
Dec. 12, 2023 (CN) 202311705885.0

(51) Int. Cl. C07C 17/281; B01J 8/00; B01J 8/08; B01J 27/10; B01J 27/122; B01J 27/128; C07C 17/383

(52) U.S. Cl. CPC C07C 17/281; B01J 8/007; B01J 8/085; B01J 27/10; B01J 27/122; B01J 27/128; C07C 17/383; B01J 2208/00752; B01J 2208/00761

(57) ABSTRACT

A preparation method and a production system for hexafluorobutadiene are provided. The method includes: step (1), the organic solution of trifluoroethylene bromide and the zinc powder are respectively successively fed into a first reactor and a second reactor equipped with an initiator, zinc powder and an organic solvent, so as to obtain a solution of trifluorovinylzinc bromide. Then, the reaction solution enters a settling device for separating excess zinc powder to obtain a solution of trifluorovinylzinc bromide containing no zinc powder, and the excess zinc powder is filtered and recycled; step (2), introducing the obtained trifluoroethylene zinc bromide solution and a pre-prepared composite catalyst solution into a third reactor to perform a coupling reaction to obtain a crude hexafluorobutadiene; step (3) purifying the crude hexafluorobutadiene product obtained in the previous step to achieve a purity level of ≥99.9%.

### CROSS-REFERENCE TO RELATED APPLICATIONS

The present application is a National Stage of International Patent Application No: PCT/CN2024/132457 filed on Nov. 15, 2024, which application claims priority to the Chinese patent application with application No. 202311705885.0, submitted to the Patent Office of the People's Republic of China on Dec. 12, 2023. The entire contents of the above-mentioned application are incorporated herein by reference.

### TECHNICAL FIELD

The present disclosure relates to the preparation of a fluorinated electronic gas, and particularly to preparation method and production system for hexafluorobutadiene.

### BACKGROUND

Hexafluorobutadiene (C₄F₆) is a colorless, liquefiable fluorinated gas with a boiling point of 5.5° C. at atmospheric pressure and a liquid density of 1.44 g/mL at 15° C. Currently, hexafluorobutadiene is mainly used as a dry etching gas for precision etching of critical dimensions (with an accuracy of 100 nm) in the production of a rectifier circuit board containing Cu and low K dielectric constant, and has better selectivity and depth to width ratio than other etching gases. Compared with octafluorocyclobutane (c-C₄F₈), the etching width to depth ratio of C₄F₆ can reach 10, while c-C₄F₈ is only 3. Therefore, C₄F₆ is suitable for an extremely narrow line width process. Therefore, C₄F₆ only etches a silicon oxide film without affecting a photoresist, silicon film, and nitride film, and has excellent etching selectivity. In addition, hexafluorobutadiene also has good environmental performance, with an ODP of 0, a GWP(100) of 290, and only exists in the atmosphere for 1.9 d, making it an extremely low greenhouse effect and environmentally friendly etching gas. Therefore, with the development of the integrated circuit industry and attention to greenhouse gases, hexafluorobutadiene, which has the best etching effect and environmentally friendly characteristics, is bound to become the leading product and be widely used in the laser etching agent market.

The preparation process of hexafluorobutadiene has been one of the hot topics in recent years, but there are not many that can truly achieve industrial production. There are mainly the following reports on the preparation of hexafluorobutadiene in the prior art:

### (1) Oxidative Coupling Process

The self-coupling route is centered around the preparation of a key intermediate trifluoroethylene zinc halide (CF₂═CFZnX), followed by self-coupling reaction in the presence of Fe³⁺ or Cu²⁺ ions to obtain hexafluorobutadiene. As disclosed in WO2006/026400, trifluorochloroethylene (CF₂═CFCl) was used as the raw material to prepare trifluorobromoethylene (CF₂═CFBr) through hydrogenation dechlorination, bromination, and dehydrobromination. Then, it was reacted with zinc powder to prepare trifluoroethylene based zinc bromide (CF₂═CFZnBr), and finally self-coupled in the presence of Fe³⁺ or Cu²⁺ ions to obtain hexafluorobutadiene. Although the material used in this method was relatively inexpensive, it involved multiple hazardous production processes, had long steps, and the intermediate product trifluoroethylene posed a risk of self-polymerization, making it unsuitable for industrial scale production.

CN104829415 disclosed a process route for preparing hexafluorobutadiene using tetrafluoroethane (HFC-134a) as raw material. Firstly, tetrafluoroethane reacted with bromine at high temperature to obtain 1,1-dibromotetrafluoroethane; then, trifluoroethylene based zinc bromide was prepared by reacting with zinc powder in a polar aprotic solvent. Finally, hexafluorobutadiene was obtained by self-coupling under a Fe³⁺ oxidant, with a total reaction yield of 47%. This reaction used an inexpensive refrigerant HFC-134a as a raw material, and the process route was relatively short, but the activity of 1,1-dibromotetrafluoroethane was low, resulting in a low yield when preparing trifluoroethylene based zinc bromide with zinc powder. This led to a high actual unit consumption and a large amount of the three wastes in this process route, and the actual industrialization prospect was low.

[Journal of Fluorine Chemistry 129 (2008) 443-446] reported the preparation of trifluoroethyl zinc chloride in a zinc chloride/tetrahydrofuran system using tetrafluoroethane (HFC-134a) as a raw material and lithium diisopropylamide (LDA) as a hydrogen extraction reagent. Hexafluorobutadiene was then obtained under the action of Cu²⁺ or Fe³⁺, with a reaction yield of 69-70%. The raw material R134a for this process was cheap and easy to obtain, with simple synthesis steps that could be synthesized in a “one pot” process. But the first step reaction used a strong base lithium diisopropylamide (LDA), which was expensive and had high risks in the production process, with low industrial feasibility.

JP2001114710 reported a process route for synthesizing hexafluorobutadiene using tetrafluoroethylene as a raw material. Firstly, tetrafluoroethylene was added with bromine to obtain 1,2-dibromotetrafluoroethane; secondly, 1,2-dibromotetrafluoroethane was rearranged under Lewis acid catalysis to obtain 1,1-dibromotetrafluoroethane; again, 1,1-dibromotetrafluoroethane reacted with zinc powder to obtain a zinc trifluoroethylene bromide reagent, and finally, a target product hexafluorobutadiene is obtained by self-coupling of the zinc trifluoroethylene bromide reagent in the presence of a Cu²⁺ or Fe³⁺ catalyst. The reaction condition of this process was relatively mild and the raw material cost was relatively low, but the overall yield of this process was low (<50%), and it used tetrafluoroethylene as a raw material, which limited its industrialization.

### (2) Dehalogenation Process

The dehalogenation process is to obtain an intermediate tetrahalogenated hexafluorobutane (XCF₂—CFX—CFX—CF₂X) through polymerization or intermolecular dehalogenation, which reacts with zinc powder in an alcohol solvent to prepare hexafluorobutadiene. U.S. Pat. No. 304,630 disclosed a for preparation method perfluorobutadiene using trifluorochloroethylene as a raw material. Firstly, trifluorochloroethylene reacted with iodine chloride (ICI) in a sealed system at 35° C.-40° C. to obtain 1,2-dichloro-1,2,2-trifluoroiodoethane. Then, it coupled with equivalent mercury under UV irradiation to obtain 1,2,3,4-tetrachloro-1,1,2,3,4,4-hexafluorobutane. Finally, 1,2,3,4-tetrachloro-1,1,2,3,4,4-hexafluorobutane was dechlorinated in an alcohol solvent under the action of zinc powder to obtain hexafluorobutadiene. This method required the use of chemical equivalents of iodine chloride and mercury to participate in the reaction, and one of the products was mercury iodide, which was highly toxic and expensive to use reagents.

CN106336342 has improved this process by using a zinc powder/acetic anhydride system for intermolecular coupling, avoiding the use of highly toxic mercury as a raw material. The process conditions were relatively mild, and the yield of the three-step reaction was high (90%). However, expensive iodine was still required as a raw material and was not suitable for large-scale production.

U.S. Pat. No. 2,894,043 disclosed the dimerization of 1,2-dichlorodifluoroethylene (CFCl═CFCl) in the presence of fluorine gas to synthesize an intermediate product 1,2,3,4-tetrachlorohexafluorobutane, which was then dechlorinated with zinc powder to obtain a target product perfluorobutadiene. This method required low-temperature conditions (−70° C.) and the use of high-risk gas F₂ during the fluorination dimerization stage. U.S. Pat. No. 2,676,193 has improved this method, but the reaction still required high temperature (300° C.) and high pressure (12 MPa) conditions, and the reaction yield was low, only 30%-40%, with many by-products and difficult product separation.

RU0118462 reported a route for synthesizing hexafluorobutadiene from trifluorochloroethylene, which avoided the use of highly toxic mercury and expensive iodine reagents. Firstly, trifluorochloroethylene was dimerized at high temperature to obtain 34% 1,2-dichlorohexafluorocyclobutane and 27% 3,4-dichlorohexafluoro-1-butene. Then these two products were separated through an efficient rectification column. 3,4-Dichlorohexafluoro-1-butene was directly dechlorinated with zinc powder to obtain a target product hexafluorobutadiene. The advantage of this method was that it only required two reaction steps to produce C₄F₆, which was relatively short. However, the separation of dimeric products required the use of highly demanding fractionation processes, which increased production costs to a certain extent, and the improved yield of target intermediates was still less than 30%.

### (3) Catalytic Coupling Process

WO2018235883 disclosed a self-coupling reaction using trifluorochloroethylene as a raw material catalyzed in the presence of palladium catalyst, phosphorus ligand, and zinc powder to obtain hexafluorobutadiene with a highest yield of 86.1%. CN116693365 disclosed the cross coupling reaction of trifluorochloroethylene and trifluoroethylene as raw materials in the presence of an active palladium catalyst and a basic compound to obtain hexafluorobutadiene, with a maximum reaction yield of 83%. Although this method has a simple process, it does not have an advantage in raw material cost as it uses expensive palladium as a catalyst.

### (4) Fluorine Gas Process

WO2007125972 disclosed a process for preparing hexafluorobutadiene using butadiene as a raw material. The main steps were as follows: firstly, 1,3-butadiene reacted with chlorine gas to produce 1,2,3,4-tetrachlorobutane; secondly, under the condition of no catalyst, using inert gas as the carrier gas, 1,2,3,4-tetrachlorobutane reacted with fluorine the gas in gas phase to generate 1,2,3,4-tetrachlorohexafluorobutane; and finally, 1,2,3,4-tetrachlorohexafluorobutane reacted with zinc powder in a solvent to obtain hexafluorobutadiene.

To address the shortcomings of the prior art, it is necessary to provide a low-cost, high-purity, safe, and continuously stable preparation method and preparation and production system for hexafluorobutadiene.

### SUMMARY

An object of the present disclosure is to provide preparation method and production system for hexafluorobutadiene, achieving safe, stable, and reliable production of hexafluorobutadiene.

The technical route of the present disclosure is as follows:

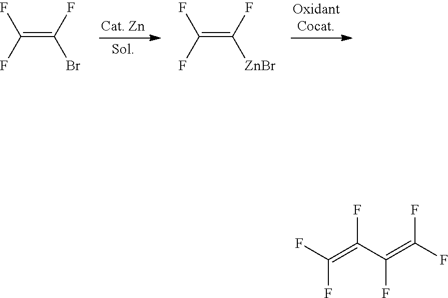

To achieve the object of the present disclosure, according to a first aspect of the present disclosure, the following technical solution is adopted:

A preparation method for hexafluorobutadiene, the preparation method includes the following steps:
Step (1), introducing an organic solution of trifluorobromoethylene and zinc powder into a first reactor containing an initiator, zinc powder, and an organic solvent, to react to obtain a trifluoroethylene zinc bromide solution; introducing the trifluoroethylene zinc bromide solution and zinc powder into a second reactor containing zinc powder, an initiator, and a first organic solvent to completely convert unreacted trifluorobromoethylene; introducing the reaction solution into a settling device to separate excess zinc powder and obtain a trifluoroethylene zinc bromide solution with zinc powder removed; and the first organic solvent is selected from a polar aprotic organic solvent;
Step (2): introducing the trifluoroethylene zinc bromide solution obtained in step (1) with zinc powder removed and a pre-prepared composite catalyst organic solution into a third reactor to perform a coupling reaction to obtain a synthetic solution comprising a crude hexafluorobutadiene; the composite catalyst organic solution consists of an oxidant, a catalytic aid, and a second organic solvent; the catalytic aid is selected from a monovalent copper salt and a ferrous salt, and the second organic solvent is selected from a polar aprotic organic solvent;
Step (3) introducing the synthetic solution obtained in step (2) into a purification system to obtain a purified hexafluorobutadiene product.

The preparation steps of the trifluorobromoethylene solution are as follows:
A. the same type of solvent (moisture ≤500 ppm) as a first reactor is accurately added to a trifluorobromoethylene solution preparation kettle;
B. then, a certain amount of trifluorobromoethylene is introduced, and the temperature inside the kettle is controlled between −10° C. to 10° C., with a mass concentration of 5%-30% for the solution. As a preferred option, the temperature inside the kettle should be controlled between 0° C.-5° C., and the mass concentration of the solution should be 15%-20%.

The specific steps for preparing the trifluoroethylene zinc bromide solution are as follows:
A. firstly, organic solvent, initiator, and zinc powder are added to the first reactor and heated up to a certain temperature under stirring conditions, at the same time, solvent, initiator, and zinc powder are added to the second reactor and heated up to a certain temperature under stirring conditions, and the overflow valve of the first reactor is opened to allow the material in the first reactor to overflow to the second reactor;
B. an organic solution of trifluorobromoethylene and zinc powder are continuously added to the first reactor, zinc powder is continuously added to the second reactor, the overflow valve of the second reactor is opened to allow the material in the second reactor to overflow or be pumped to a settling device to obtain a trifluoroethylene zinc bromide solution after removing the zinc powder. The settling device includes multi-stage settling tanks, preferably using a two-stage settling tank. The trifluoroethylene zinc bromide solution prepared in step (1) enters a first settling tank through overflow or pump transportation. The overflow valve or pump of the first settling tank is opened to allow the material in the first settling tank to overflow or pump to a second settling tank, obtaining a trifluoroethylene zinc bromide solution without zinc powder residue. The settling tank can use a settling pressure filtration tank to settle excess zinc powder to a settling tank baffle, obtaining a trifluoroethylene zinc bromide solution without zinc powder residue. After pressure filtration, the zinc powder in the settling tank can be directly used for the preparation reaction of trifluoroethylene zinc bromide solution.

The initiator is selected from any one or more of bromomethane, 1,2-dibromoethane, elemental iodine, trimethylchlorosilane, and trifluoroethylene zinc bromide solution. The molar ratio of the feed rate of trifluorobromoethylene (mol/h) to the amount of base initiator (mol) is 1:(1-100).

As a preferred option, the initiator is selected from one of 1,2-dibromoethane, elemental iodine, and trifluoroethylene zinc bromide reagent solution. The molar ratio of the feed rate (mol/h) of trifluorobromoethylene to the amount of base initiator (mol) is 1:(1-50). Further preferred, the initiator is a trifluoroethylene zinc bromide reagent solution.

In the process of preparing the trifluoroethylene zinc bromide solution, the ratio of the feed rate value of the trifluorobromoethylene organic solution in kg/h to the mass value of the first reactor base material in kg is 1:(10-100); and the ratio of the feed rate value of the organic solution of trifluorobromoethylene in kg/h to the mass value of the second reactor base material in kg is 1:(5-100).

As a preferred option, the ratio of the feed rate value of the trifluorobromoethylene organic solution in kg/h to the mass value of the first reactor base material in kg is 1:(10-50); and the ratio of the feed rate value of the organic solution of trifluorobromoethylene in kg/h to the mass value of the second reactor base material in kg is 1:(10-50).

In the process of preparing the trifluoroethylene zinc bromide solution, the molar ratio of trifluorobromoethylene to the feed rate of zinc powder in the first reactor is 1:(1.0-5.0), and the molar ratio of trifluorobromoethylene to the feed rate of zinc powder in the second reactor is 1:(0.1-2.0).

As a preferred option, in the reaction process, the molar ratio of trifluorobromoethylene to the feed rate of zinc powder in the first reactor is 1:(1.0-3.0), and the molar ratio of trifluorobromoethylene to the feed rate of zinc powder in the second reactor is 1:(0.1-1.0).

During the preparation of trifluoroethylene zinc bromide solution in step (1), the mesh size of the zinc powder ranges from 100 to 500 meshes.

As a preferred option, zinc powder has a mesh size of 200-400.

In the process of preparing the trifluoroethylene zinc bromide solution in step (1), the organic solvent is selected from a polar aprotic organic solvent; the polar aprotic organic solvent is selected from one, two or more combinations of tetrahydrofuran, N, N-dimethylformamide, N, N-dimethylacetamide, hexamethylphosphoramide, dimethyl sulfoxide, and N-methylpyrrolidone, and the moisture content of the polar aprotic organic solvent is ≤500 ppm.

As a preferred option, the polar aprotic organic solvent in step (1) is selected from one, two or more combinations of N, N-dimethylformamide, N, N-dimethylacetamide, and dimethyl sulfoxide, and the moisture content of the polar aprotic organic solvent is ≤200 ppm.

In the process of preparing the trifluoroethylene zinc bromide solution in step (1), the temperature range of the first reactor is 60° C.-120° C., and the temperature range of the second reactor is 60° C.-90° C.

As a preferred option, the temperature range of the first reactor is 60° C.-90° C., and the temperature range of the second reactor is 60° C.-70° C.

In the process of preparing hexafluorobutadiene in step (2), the oxidant is selected from any one or more of sodium peroxide, potassium peroxide, sodium perborate, sodium persulfate, potassium persulfate, ammonium persulfate, and di-tert-butyl peroxide; the catalytic aid is selected from any one or more of cuprous chloride, cuprous bromide, cuprous iodide, ferrous chloride, and ferrous bromide; and the polar aprotic organic solvent is selected from any one or more of tetrahydrofuran, N, N-dimethylformamide, N, N-dimethylacetamide, hexamethylphosphoramide, dimethyl sulfoxide, and N-methylpyrrolidone.

As a preferred option, in the process of preparing hexafluorobutadiene in step (2), the oxidant is selected from ammonium perborate, sodium persulfate, and potassium persulfate; and the aid is selected from any one or more of cuprous iodide and ferrous chloride. The polar aprotic organic solvent is selected from any one or more of N, N-dimethylformamide, N, N-dimethylacetamide, and dimethyl sulfoxide.

In the process of preparing hexafluorobutadiene in step (2), the molar ratio of trifluoroethylene based zinc bromide to oxidant is 1:(1.0-3.0). The molar ratio of trifluoroethylene based zinc bromide to catalytic aid in the trifluoroethylene based zinc bromide solution is 1:(0.01-0.2).

As a preferred option, the molar ratio of trifluoroethylene zinc bromide to oxidant is 1:(1.0-1.5). The molar ratio of trifluoroethylene zinc bromide to catalytic aid in the trifluoroethylene zinc bromide solution is 1:(0.01-0.05).

In the process of preparing hexafluorobutadiene in step (2), the temperature of the coupling reaction is −10° C. to 50° C., the reaction pressure is 0 MPa-0.5 MPa, and the residence time in the reactor is 10 s-600 s.

As a preferred option, the temperature of the coupling reaction is 0° C.-10° C., the reaction pressure is 0 MPa-0.2 MPa, and the residence time in the reactor is 50 s-300 S.

In step (3), the preferred purification system is a combination of a distillation device and a rectification device. After the reaction is completed, the reaction solution enters the distillation unit to quickly evaporate the product, which is then rectified to obtain a high-purity hexafluorobutadiene product.

The content of difficult-to-separate impurities trifluorobromoethylene in the crude hexafluorobutadiene obtained by distillation is ≤0.1%, the content of heptafluorobutene is ≤0.01%, and the purity of the product after distillation is ≥99.9%.

According to a second aspect of the present disclosure, the following technical solution is adopted:
A preparation and production system for preparing hexafluorobutadiene, which is used for the above-mentioned preparation method, and includes a trifluoroethylene zinc bromide solution preparation unit, a hexafluorobutadiene preparation unit, a zinc powder filtration unit, a distillation unit, and a rectification unit;
The trifluoroethylene zinc bromide solution preparation unit includes a trifluorobromoethylene organic solution feeding device, a zinc powder feeding device, a solvent and initiator feeding device, a first reactor, and a second reactor; wherein the trifluorobromoethylene organic solution feeding device, the zinc powder feeding device, and the solvent and initiator feeding device are connected to the first reactor for feeding; the zinc powder feeding device, and the solvent and initiator feeding device are connected to the second reactor for feeding; the first reactor is connected to the second reactor and is used to output the trifluoroethylene zinc bromide solution obtained from the reaction in the first reactor to the second reactor; the upper parts of the first reactor and the second reactor are connected to a vacuum and high-purity nitrogen device through a condenser, respectively; and an outlet of the second reactor is connected to a zinc powder settling device;
The hexafluorobutadiene preparation unit includes a trifluoroethylene zinc bromide solution feeding device, a composite catalyst solution feeding device, and a third reactor, and the trifluoroethylene zinc bromide solution feeding device and the composite catalyst solution feeding device are connected to an inlet end of the third reactor;
The distillation unit includes a hexafluorobutadiene synthetic solution feeding device, a distillation device, and a product collection device;
The rectification unit includes a crude hexafluorobutadiene feeding device, a rectification tower, a front distillate storage tank, a product collection tank, and a rectification residue storage tank.

The material of each equipment in the trifluoroethylene zinc bromide solution preparation unit is selected from one of enamel glass, carbon steel, 316L, and fluorine resin lined carbon steel; the equipment material of the zinc powder filtration unit is selected from one of carbon steel, 304, and 316L; and the equipment material of the distillation unit is selected from one of enamel glass, 304, and 316L.

Furthermore, the first reactor and the second reactor are equipped with stirring devices, and the type of the stirring blade is selected from a propulsion stirring blade; the distillation device is equipped with a stirring device, and the type of the stirring blade is selected from an anchor stirring blade.

The beneficial effects of the present disclosure are:
(1) In the preparation step of trifluoroethylene zinc bromide, a two-stage series reactor is used, which not only achieves almost complete conversion of the raw material trifluorobromoethylene, improves the utilization rate of the raw material, but also reduces the pressure for subsequent product distillation purification, which is conducive to improving the purity of the product.
(2) In the process of preparing hexafluorobutadiene by oxidative coupling, inexpensive peroxides are used as oxidants, and catalytic amounts of copper or iron salts are used as aids. Compared with traditional processes that use equivalent amounts of copper or iron salts, this not only significantly reduces raw material costs, but also reduces the emission of heavy metal waste solids and alleviates environmental pressure. At the same time, using inorganic peroxides as oxidants, compared to metal chlorides as oxidants, can reduce the production of difficult to separate impurity fluorinated olefins, which is beneficial for improving product purity.

### BRIEF DESCRIPTION OF THE DRAWINGS

FIG. 1 is a schematic diagram of the trifluoroethylene based zinc bromide solution preparation unit and the zinc powder filtration unit in the embodiment of the present disclosure.

FIG. 2 is a schematic diagram of the hexafluorobutadiene preparation unit and distillation unit in the embodiment of the present disclosure.

FIG. 3 is the chromatographic analysis spectrum of a hexafluorobutadiene product prepared in Example 15 of the present disclosure.

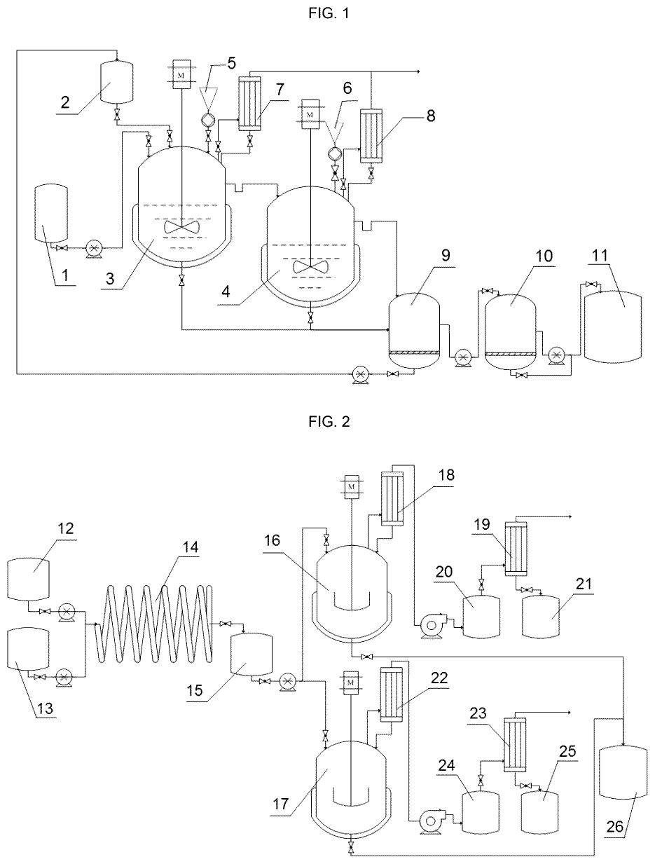

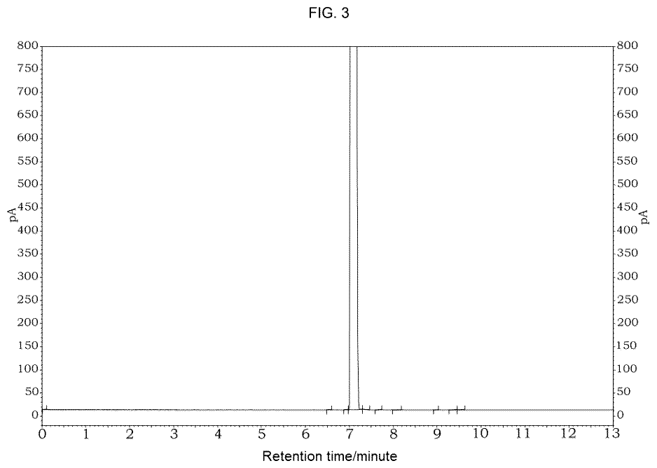

Among them, the above figures include the following reference numerals:
1: trifluorobromoethylene solution storage tank; 2: initiator or solvent storage tank; 3: first reactor; 4: second reactor; 5: first zinc powder storage tank; 6: second zinc powder storage tank; 7: first condenser; 8: second condenser; 9: first settling tank, 10: second settling tank; 11: first trifluoroethylene zinc bromide solution storage tank; 12: second trifluoroethylene zinc bromide solution storage tank; 13: composite catalyst solution storage tank; 14: third reactor; 15: hexafluorobutadiene synthesis buffer tank; 16: first distillation device; 17: second distillation device; 18: third condenser; 19: fourth condenser; 20: first hexafluorobutadiene buffer tank; 21: first hexafluorobutadiene crude product tank; 22: fifth condenser; 23: sixth condenser; 24: second hexafluorobutadiene buffer tank; 25: second hexafluorobutadiene crude product tank; 26: distillation residue storage tank.

### DETAILED DESCRIPTION OF THE EMBODIMENTS

The present disclosure will be further explained with specific embodiments, but the present disclosure is not limited to these specific embodiments. Those skilled in the art should recognize that the present disclosure encompasses all alternative, improved, and equivalent solutions that may be included within the scope of the claims.

Firstly, the production system for preparing hexafluorobutadiene according to the present disclosure will be described with reference to the accompanying drawings.

The preparation and production system for hexafluorobutadiene includes a trifluoroethylene zinc bromide solution preparation unit, a hexafluorobutadiene preparation unit, a zinc powder filtration unit, a distillation unit, and a rectification unit.

The trifluoroethylene zinc bromide solution preparation unit includes a trifluorobromoethylene organic solution feeding device, a zinc powder feeding device, a solvent and initiator feeding device, a first reactor 3, and a second reactor 4.

The trifluorobromoethylene organic solution feeding device includes a trifluorobromoethylene solution storage tank 1, and the solvent and initiator feeding device includes an initiator or solvent storage tank 2, which are respectively fed to the corresponding feeding positions in the upper part of the first reactor 3 through pumping or gravity. The zinc powder feeding device includes a first zinc powder storage tank 5, which uses gravity feeding. The first reactor 3 and the second reactor 4 are connected to output the trifluoroethylene zinc bromide solution obtained from the reaction in the first reactor 3 to the second reactor 4. The second reactor 4 is also connected to the zinc powder feeding device, which can use a second zinc powder storage tank 6 for gravity feeding. The upper parts of the first reactor 3 and the second reactor 4 are connected to vacuum devices through a first condenser 7 and a second condenser 8, respectively; the outlet of the second reactor 4 is connected to the zinc powder filtration unit; the zinc powder filtration unit can use two-stage settling tanks, namely a first settling tank 9 and a second settling tank 10, and the output of the second settling tank 10 is connected to a first trifluoroethylene based zinc bromide solution storage tank 11, which can be used as a material buffering link for production.

The hexafluorobutadiene preparation unit includes a trifluoroethylene zinc bromide solution feeding device, a composite catalyst solution feeding device, and a third reactor 14, and the third reactor 14 is a tubular reactor, and the trifluoroethylene zinc bromide solution feeding device and the composite catalyst solution feeding device are connected to the inlet end of the third reactor; the trifluoroethylene zinc bromide solution feeding device includes a second trifluoroethylene zinc bromide solution storage tank 12, which can be the same as a first trifluoroethylene zinc bromide solution storage tank 11, and the trifluoroethylene zinc bromide solution feeding device is pumped to the third reactor 14 for feeding. The composite catalyst solution feeding device includes a composite catalyst solution storage tank 13, which is pumped to the third reactor 14 for feeding.

The distillation unit includes a hexafluorobutadiene synthetic solution feeding device, a distillation device, and a product collection device. The outlet end of the third reactor 14 can be connected to a hexafluorobutadiene synthetic solution buffer tank 15.

The distillation unit includes a hexafluorobutadiene synthetic solution feeding device, a distillation device, and a product collection device.

The hexafluorobutadiene synthetic solution feeding device includes a hexafluorobutadiene synthetic solution buffer tank 15, which is pumped to feed two parallel purification systems. A first purification system includes a first distillation device 16, which is connected to a first hexafluorobutadiene buffer tank 20 through a third condenser 18, the first hexafluorobutadiene buffer tank 20 is connected to a first hexafluorobutadiene crude product tank 21 through a fourth condenser 19; a second purification system includes a second distillation unit 17, which is connected to a second hexafluorobutadiene buffer tank 24 through a fifth condenser 22, the second hexafluorobutadiene buffer tank 24 is connected to a second hexafluorobutadiene crude product tank 25 through a sixth condenser 23, and the second hexafluorobutadiene crude product tank 25 is then connected to a rectification unit. The reference number 26 in the attached figure is a distillation residue storage tank.

The following embodiments utilize the above-mentioned production system.

### Embodiment 1

(1) in the first reactor of 500 L of 316L material, 300 kg of N, N-dimethylformamide solution with a mass fraction of 25% trifluoroethylene zinc bromide was added, stirring was started, 65 kg of zinc powder was added, and the reactor was heated up to 80° C.;
(2) in the second reactor of 500 L of 316L material, 300 kg of N, N-dimethylformamide solution with a mass fraction of 25% trifluoroethylene zinc bromide was added, stirring was started, 32.5 kg of zinc powder was added, and the reactor was heated up to 60° C.;
(3) N, N-dimethylformamide solution of trifluorobromoethylene (mass fraction of 20%) was added to the first reactor, and the flow rate was controlled at 15 kg/h; and simultaneously, zinc powder (325 meshes, 1.3 kg/h) was added to the first reactor through the solid feeding device;
(4) the overflow valve connecting the first reactor and the second reactor were opened, and when the liquid level of the material in the first reactor exceeded the height of the overflow port, it entered the second reactor; simultaneously, zinc powder (325 meshes, 0.35 kg/h) was added to the second reactor through the solid feeding device;
(5) the overflow valves of the second reactor and the first settling tank were opened, and when the liquid level of the material in the second reactor exceeded the height of the overflow port, it entered the first settling tank and settled the excess zinc powder to the baffle;
(6) the connecting valve between the first settling tank and the second settling tank were opened, when the liquid level of the material in the first settling tank exceeded the height of the valve outlet, the material transfer pump was opened to transfer the material to the second settling tank and settle the residual zinc powder to the baffle; and
(7) the connecting valve between the second settling tank and the storage tank of the trifluoroethylene zinc bromide solution were added, when the liquid level of the material in the second settling tank exceeded the height of the valve outlet, the material transfer pump was opened to transfer the material to the storage tank. Running for 72 hours, samples were taken from the storage tank of the trifluoroethylene zinc bromide solution for analysis. The results showed that the normalized content of trifluorobromoethylene was 0.005%, the mass fraction of trifluoroethylene zinc bromide was 25.1% (nuclear magnetic fluorine spectrum internal standard method), the theoretical mass fraction was 26%, and the average yield was 96.5%.

### Embodiment 2

The operation of the present embodiment was the same as that of Embodiment 1, with the only difference being that: the N, N-dimethylformamide solution of trifluoroboromoethylene (mass fraction of 20%) with a flow rate of 15 kg/h was replaced by the N, N-dimethylformamide solution (mass fraction of 20%) with a flow rate of 20 kg/h; adding zinc powder (325 meshes, 1.3 kg/h) to the first reactor was replaced by adding zinc powder (325 meshes, 1.8 kg/h) to the first reactor; and adding zinc powder (325 meshes, 0.35 kg/h) to the second reactor was replaced by adding zinc powder (325 meshes, 0.48 kg/h) to the second reactor, and other conditions remained unchanged. Running for 72 hours, samples were taken from the storage tank of the trifluoroethylene zinc bromide solution for analysis. The results showed that the normalized content of trifluorobromoethylene was 0.008%, the mass fraction of trifluoroethylene zinc bromide was 24.4% (nuclear magnetic fluorine spectrum internal standard method), the theoretical mass fraction was 26%, and the average yield was 93.8%.

### Embodiment 3

The operation of the present embodiment was the same as that of Embodiment 1, with the only difference being that: adding zinc powder (325 meshes, 0.35 kg/h) to the second reactor was replaced by adding zinc powder (325 meshes, 0.20 kg/h) to the second reactor.

Other conditions remained unchanged. Running for 72 hours, samples were taken from the storage tank of the trifluoroethylene zinc bromide solution for analysis. The results showed that the normalized content of trifluorobromoethylene was 0.006%, the mass fraction of trifluoroethylene zinc bromide was 24.7% (nuclear magnetic fluorine spectrum internal standard method), the theoretical mass fraction was 26%, and the average yield was 95.0%.

### Embodiment 4

The operation of the present embodiment was the same as that of Embodiment 1, with the only difference being that: the mesh size of the zinc powder has been replaced from 325 meshes to 500 meshes, and other conditions remained unchanged. Running for 72 hours, samples were taken from the storage tank of the trifluoroethylene zinc bromide solution for analysis. The results showed that the normalized content of trifluorobromoethylene was 0.01%, the mass fraction of trifluoroethylene zinc bromide was 22.2% (nuclear magnetic fluorine spectrum internal standard method), the theoretical mass fraction was 26%, and the average yield was 85.3%.

### Embodiment 5

The operation of the present embodiment was the same as that of Embodiment 1, with the only difference being that: the temperature of the first reactor has been replaced from 80° C. to 60° C., and other conditions remained unchanged. After running for 72 hours, a sample of the trifluoroethylene based zinc bromide solution storage tank is taken for analysis, running for 72 hours, samples were taken from the storage tank of the trifluoroethylene zinc bromide solution for analysis. The results showed that the normalized content of trifluorobromoethylene was 0.012%, the mass fraction of trifluoroethylene zinc bromide was 21.7% (nuclear magnetic fluorine spectrum internal standard method), the theoretical mass fraction was 26%, and the average yield was 83.3%.

### Embodiment 6

The operation of the present embodiment was the same as that of Embodiment 1, with the only difference being that: the temperature of the second reactor has been replaced from 60° C. to 70° C., and other conditions remained unchanged. After running for 72 hours, a sample of the trifluoroethylene based zinc bromide solution storage tank is taken for analysis, running for 72 hours, samples were taken from the storage tank of the trifluoroethylene zinc bromide solution for analysis. The results showed that the normalized content of trifluorobromoethylene was 0.005%, the mass fraction of trifluoroethylene zinc bromide was 24.9% (nuclear magnetic fluorine spectrum internal standard method), the theoretical mass fraction was 26%, and the average yield was 95.8%.

### Embodiment 7

The operation of the present embodiment was the same as that of Embodiment 1, with the only difference being that: adding the N, N-dimethylformamide solution with a mass fraction of 25% trifluoroethylene zinc bromide to the first and second reactors was replaced by adding the N, N-dimethylacetamide solution with a mass fraction of 25% trifluoroethylene zinc bromide to the first and second reactors. Adding the N, N-dimethylformamide solution of trifluorobromoethylene to the first reactor (mass fraction of 20%) was replaced by adding the N, N-dimethylacetamide solution of trifluorobromoethylene to the first reactor (mass fraction of 20%), and other conditions remained unchanged. Running for 72 hours, samples were taken from the storage tank of the trifluoroethylene zinc bromide solution for analysis. The results showed that the normalized content of trifluorobromoethylene was 0.007%, the mass fraction of trifluoroethylene zinc bromide was 23.9% (nuclear magnetic fluorine spectrum internal standard method), the theoretical mass fraction was 26%, and the average yield was 91.9%.

### Embodiment 8

The operation of the present embodiment was the same as that of Embodiment 1, with the only difference being that: the zinc powder was replaced by the zinc powder recovered from the first settling tank, and other conditions remain unchanged. After running for 72 hours, a sample of the trifluoroethylene based zinc bromide solution storage tank is taken for analysis, running for 72 hours, samples were taken from the storage tank of the trifluoroethylene zinc bromide solution for analysis. The results showed that the normalized content of trifluorobromoethylene was 0.006%, the mass fraction of trifluoroethylene zinc bromide was 24.3% (nuclear magnetic fluorine spectrum internal standard method), the theoretical mass fraction was 26%, and the average yield was 93.5%.

### Comparative Embodiment 1

The trifluoroethylene based zinc bromide solution was prepared by a single reactor reaction, with the following operational steps:
(1) In the first reactor of 500 L of 316L material, 300 kg of N, N-dimethylformamide solution with a mass fraction of 25% trifluoroethylene zinc bromide was added, stirring was started, 65 kg of zinc powder was added, and the reactor was heated up to 80° C.;
(2) N, N-dimethylformamide solution of trifluorobromoethylene (mass fraction of 20%) was added to the first reactor, and the flow rate was controlled at 15 kg/h; and simultaneously, zinc powder (325 meshes, 1.3 kg/h) was added to the first reactor through the solid feeding device;
(3) the overflow valves of the first reactor and the first settling tank were opened, and when the liquid level of the material in the second reactor exceeded the height of the overflow port, it entered the first settling tank and settled the excess zinc powder to the baffle;
(4) the connecting valve between the first settling tank and the second settling tank were opened, when the liquid level of the material in the first settling tank exceeded the height of the valve outlet, the material transfer pump was opened to transfer the material to the second settling tank and settle the residual zinc powder to the baffle;
(5) the connecting valve between the second settling tank and the storage tank of the trifluoroethylene zinc bromide solution were added, when the liquid level of the material in the second settling tank exceeded the height of the valve outlet, the material transfer pump was opened to transfer the material to the storage tank. Running for 72 hours, samples were taken from the storage tank of the trifluoroethylene zinc bromide solution for analysis. The results showed that the normalized content of trifluorobromoethylene was 1.56%, the mass fraction of trifluoroethylene zinc bromide was 20.2% (nuclear magnetic fluorine spectrum internal standard method), the theoretical mass fraction was 26%, and the average yield was 77.8%.

### Embodiment 9

(1) 300 kg of N, N-dimethylformamide was added to a 500 L of an enamel glass reaction vessel, ammonium persulfate (40 kg, 175 mol) and cuprous iodide (0.67 kg, 3.5 mol) were added under stirring conditions, and the internal temperature was maintained at 0° C.-5° C. to complete the preparation of the oxidant solution, with an oxidant concentration of 0.51 mol/kg and an aid concentration of 0.01 mol/kg.
(2) The oxidant solution (a flow rate of 40 kg/h, equivalent to ammonium persulfate flow rate of 20.4 mol/h, and cuprous iodide flow rate of 0.4 mol/h) and the trifluoroethylene zinc bromide solution (a mass fraction of 25%, a flow rate of 16.8 kg/h, equivalent to a trifluoroethylene zinc bromide flow rate of 18.6 mol/h) prepared above were added into a spiral reaction tube made of 316 L material (total length of 10 meters, inner diameter of 8 mm), the internal temperature was controlled at 0° C.-5° C., and the pressure inside the reaction tube was controlled to be 0.05 MPa.
(3) The synthetic solution was introduced into a distillation device A or B from the outlet of the reaction tube. The temperature inside the kettle was 60° C., and the vacuum degree inside the kettle was 0.1 MPa. The crude hexafluorobutadiene was collected by condensation from the vacuum pump, and the condenser temperature was −15° C. After running for 72 hours, the result showed that 107.6 kg of the crude hexafluorobutadiene was collected from the crude product tank after the pump, with a main content of 95.33% (the content of other impurities was shown in Table 1 below). Converted to 102.6 kg on a hundredweight basis, with a theoretical yield of 108.5 kg and an average yield of 94.6%.

### Embodiment 10

The operation of the present embodiment was the same as that of Embodiment 9, with the only difference being that: the oxidant solution (flow rate of 60 kg/h, equivalent to ammonium persulfate flow rate of 30.6 mol/h, and cuprous iodide flow rate of 0.6 mol/h) and trifluoroethylene zinc bromide solution (mass fraction of 25%, flow rate of 25.2 kg/h, equivalent to trifluoroethylene zinc bromide flow rate of 27.9 mol/h) prepared above were added. Other conditions remained unchanged and run for 72 h. The result showed that 156.1 kg of the crude hexafluorobutadiene was collected from the crude product tank after the pump, with a main content of 95.81% (the content of other impurities was shown in Table 1 below). Converted to 149.5 kg on a hundredweight basis, with a theoretical yield of 162.8 kg and an average yield of 91.8%.

### Embodiment 11

The operation of the present embodiment was the same as that of Embodiment 9, with the only difference being that: in step (1), ammonium persulfate (40 kg, 0.175 kmol) was replaced by potassium persulfate (40 kg, 0.148 mmol), the oxidant concentration was 0.43 mol/kg, and the aid concentration was 0.01 mol/kg.

In step (2), replace the oxidant solution (flow rate of 40 kg/h, equivalent to ammonium persulfate flow rate of 20.4 mol/h, and cuprous iodide flow rate of 0.4 mol/h) was replaced by the oxidant solution (flow rate of 47.4 kg/h, equivalent to potassium persulfate flow rate of 20.4 mol/h, and cuprous iodide flow rate of 0.4 mol/h), and other conditions remained unchanged. After running for 72 hours, the result showed that 104.3 kg of the crude hexafluorobutadiene was collected from the crude product tank after the pump, with a main content of 93.80% (the content of other impurities was shown in Table 1 below). Converted to 97.8 kg on a hundredweight basis, with a theoretical yield of 108.5 kg and an average yield of 90.1%.

### Embodiment 12

The operation of the present embodiment was the same as that of Embodiment 9, with the only difference being that: in step (1), cuprous iodide (0.67 kg, 3.5 mol) was replaced by ferrous chloride (0.70 kg, 3.5 mol). Other conditions remained unchanged. After running for 72 hours, the result showed that 109.4 kg of the crude hexafluorobutadiene was collected from the crude product tank after the pump, with a main content of 94.68% (the content of other impurities was shown in Table 1 below). Converted to 103.6 kg on a hundredweight basis, with a theoretical yield of 108.5 kg and an average yield of 95.5%.

### Embodiment 13

The operation of the present embodiment was the same as that of Embodiment 9, with the only difference being that: in step (1), cuprous iodide (0.67 kg, 3.5 mol) was replaced by cuprous iodide (1.33 kg, 7.0 mol) to complete the preparation of the oxidant solution, and the aid concentration was replaced from 0.01 mol/kg to 0.02 mol/kg. The flow rate of cuprous iodide in step (2) was replaced from 0.4 mol/h to 0.8 mol/h. Other conditions remained unchanged. After running for 72 hours, the result showed that 107.4 kg of the crude hexafluorobutadiene was collected from the crude product tank after the pump, with a main content of 95.02% (the content of other impurities was shown in Table 1 below). Converted to 102.0 kg on a hundredweight basis, with a theoretical yield of 108.5 kg and an average yield of 94.0%.

### Embodiment 14

The operation of the present embodiment was the same as that of Embodiment 9, with the only difference being that maintaining an internal temperature of 0° C.-5° C. was replaced by maintaining an internal temperature of 5° C.-10° C., and in step (2), controlling an internal temperature of 0° C.-5° C. is replaced by 5° C.-10° C., and other conditions remained unchanged. After running for 72 hours, the result showed that 107.1 kg of the crude hexafluorobutadiene was collected from the crude product tank after the pump, with a main content of 94.25% (the content of other impurities was shown in Table 1 below). Converted to 100.9 kg on a hundredweight basis, with a theoretical yield of 108.5 kg and an average yield of 93.0%.

### Comparative Embodiment 2

The operation of the present embodiment was the same as that of Embodiment 9, with the only difference being that during the oxidant solution preparation process in step (1), the aid cuprous iodide was not added, and other conditions remained unchanged. After running for 72 hours, the result showed that 10.7 kg of the crude hexafluorobutadiene was collected from the crude product tank after the pump, with a main content of 66.51% (the content of other impurities was shown in Table 1 below). Converted to 7.1 kg on a hundredweight basis, with a theoretical yield of 108.5 kg and an average yield of 6.5%.

### Comparative Embodiment 3

The operation of the present embodiment was the same as that of Embodiment 9, with the only difference being that in step (3), the hexafluorobutadiene synthetic solution was performed a kettle type intermittent distillation, and the synthetic solution running for 72 h was fed into the distillation device at once, while other conditions remained unchanged. The result showed that 98.3 kg of the crude hexafluorobutadiene was collected from the crude product tank after the pump, with a main content of 93.85% (the content of other impurities was shown in Table 1 below). Converted to 92.3 kg on a hundredweight basis, with a theoretical yield of 108.5 kg and an average yield of 85.1%.

### Comparative Embodiment 4

The operation of the present embodiment was the same as that of Embodiment 9, with the only difference being that during the oxidant solution preparation process in step (1), an equimolar amount of an iron salt oxidant was used instead of a persulfate oxidant, as follows:
(1) 300 kg of N, N-dimethylformamide was added to a 500 L of an enamel glass reaction vessel, ferric chloride (28.4 kg, 0.175 kmol) was added under a stirring condition, and the internal temperature was maintained at 0° C.-5° C. to complete the preparation of the oxidant solution, with an oxidant concentration of 0.53 mol/kg.
(2) the oxidant solution (flow rate of 38.5 kg/h, equivalent to 20.4 mol/h of ferric chloride) and the trifluoroethylene zinc bromide solution (a mass fraction of 25%, a flow rate of 16.8 kg/h, equivalent to 18.6 mol/h of trifluoroethylene based zinc bromide) prepared above were added into a spiral reaction tube of 316 L material (total length of 10 meters, inner diameter of 8 mm), the internal temperature was controlled at 0° C.-5° C., and the pressure inside the reaction tube was controlled to be 0.05 MPa.

Step (3) was the same as Embodiment 9, which was run for 72 h. The result showed that 102.7 kg of the crude hexafluorobutadiene was collected from a crude product tank after a pump, with a main content of 88.52% (the content of other impurities was shown in Table 1 below). Converted to 90.9 kg on a hundredweight basis, with a theoretical yield of 108.5 kg and an average yield of 83.8%.

TABLE 1
Composition of the crude hexafluorobutadiene prepared
in Embodiments 9-14 and Comparative Embodiments 2-4

| | | | N, | | | | |
|---|---|---|---|---|---|---|---|
| | | | N-dimethyl- | | | C₄F₆ | |
| Batch | C₄F₆/% | C₂F₃H/% | formamide/% | C₂F₃Br/% | C₄F₇H/% | Dimer | Other |
| Embodiment 9 | 95.33 | 2.9445 | 1.56 | 0.056 | 0.0083 | 0.0242 | 0.077 |
| Embodiment 10 | 95.81 | 2.1488 | 1.83 | 0.075 | 0.0062 | 0.0390 | 0.091 |
| Embodiment 11 | 93.80 | 4.0014 | 2.05 | 0.058 | 0.0086 | 0.0270 | 0.055 |
| Embodiment 12 | 94.68 | 3.6869 | 1.48 | 0.032 | 0.0049 | 0.0322 | 0.084 |
| Embodiment 13 | 95.02 | 3.222 | 1.50 | 0.090 | 0.0092 | 0.0488 | 0.110 |
| Embodiment 14 | 94.25 | 3.7545 | 1.72 | 0.095 | 0.0075 | 0.0430 | 0.130 |
| Comparative | 66.51 | 22.1374 | 10.70 | 0.038 | 0.028 | 0.0066 | 0.580 |
| Embodiment 2 | | | | | | | |
| Comparative | 93.85 | 2.568 | 2.20 | 0.420 | 0.103 | 0.0690 | 0.790 |
| Embodiment 3 | | | | | | | |
| Comparative | 88.52 | 7.4105 | 1.67 | 1.420 | 0.207 | 0.0775 | 0.695 |
| Embodiment 4 | | | | | | | |

### Embodiment 15

The crude hexafluorobutadiene prepared in Embodiment 9 was used as a raw material for a rectification experiment. The rectification tower parameters and rectification parameters were shown in Tables 2 and 3 below:

TABLE 2
Rectification tower parameters

| | | | | Theoretical |
|---|---|---|---|---|
| Tower kettle | Tower inner | Tower body | | number of |
| volume | diameter | height | Filler | tower plates |
| 100 L | 80 mm | 8 h | CY700 | 30 |

TABLE 3
Rectification process parameters

| External | | Condenser | |
|---|---|---|---|
| temperature of | Tower kettle | medium | Reflux |
| tower kettle | pressure | temperature | ratio |
| 60 °C | 0.1 MPa-0.3 MPa | 0 °C-5 °C | 5:1-10:1 |

Rectification feed 100 kg, front distillate 22.6 kg, product 65.7 kg, kettle residue 10.5 kg. The product purity was 99.9908%, the single batch rectification yield was 65.7%, and the material balance rate was 98.8%.

| Retention | | Peak | Material |
|---|---|---|---|
| time | Peak area | area/% | name |
| 0.043 | 3489 | 0.0007 | |
| 6.521 | 5862 | 0.0013 | |
| 6.928 | 5132 | 0.0011 | |
| 7.028 | 465490499 | 99.9908 | C₄F₆ |
| 7.317 | 2571 | 0.0006 | |
| 7.667 | 7791 | 0.0017 | |
| 8.071 | 5010 | 0.0011 | |
| 8.977 | 2135 | 0.0005 | |
| 9.383 | 5026 | 0.0011 | |
| 9.539 | 5888 | 0.0013 | |
| Total | 465533403 | 100.0000 | |

1. A preparation method for hexafluorobutadiene, wherein the preparation method includes the following steps:
step (1), introducing an organic solution of trifluorobromoethylene and zinc powder into a first reactor containing an initiator, zinc powder, and an organic solvent, to react to obtain a trifluoroethylene zinc bromide solution; introducing the trifluoroethylene zinc bromide solution and zinc powder into a second reactor containing zinc powder, an initiator, and a first organic solvent to completely convert unreacted trifluorobromoethylene; introducing the reaction solution into a settling device to separate excess zinc powder and obtain a zinc bromide solution with zinc powder removed; and the first organic solvent is selected from a polar aprotic organic solvent;
step (2): introducing the trifluoroethylene zinc bromide solution obtained in the step (1) with zinc powder removed and a pre-prepared composite catalyst organic solution into a third reactor to perform a coupling reaction to obtain a synthetic solution comprising a crude hexafluorobutadiene; the composite catalyst organic solution consists of an oxidant, a catalytic aid, and a second organic solvent; the catalytic aid is selected from a monovalent copper salt and a ferrous salt, and the second organic solvent is selected from a polar aprotic organic solvent;
step (3) introducing the synthetic solution obtained in the step (2) into a purification system to obtain purified hexafluorobutadiene.

2. The preparation method for hexafluorobutadiene according to claim 1, wherein the initiator is selected from any one or more of bromomethane, 1,2-dibromoethane, elemental iodine, trimethylchlorosilane, and a trifluoroethylene zinc bromide reagent solution.

3. The preparation method for hexafluorobutadiene according to claim 1, wherein the initiator is selected from any one or more of the 1,2-dibromoethane, the trimethylchlorosilane, and the trifluoroethylene zinc bromide reagent solution, and the ratio of the feed rate of the trifluorobromoethylene in mol/h to the molar value of the amount of the base initiator is 1:(1-100).

4. The preparation method for hexafluorobutadiene according to claim 1, wherein the mass concentration of an organic solution of the trifluorobromoethylene is 5%-30%; during the reaction process, the ratio of the feed rate of the organic solution of the trifluorobromoethylene in kg/h to the mass value of the first reactor base material in kg is 1:(10-100); the ratio of the feed rate value of the organic solution of the trifluorobromoethylene in kg/h to the mass value of the second reactor base material in kg is 1:(5-100); the molar ratio of the trifluorobromoethylene to the feed rate of the zinc powder in the first reactor is 1:(1.0-5.0), and the molar ratio of the trifluorobromoethylene to the feed rate of the zinc powder in the second reactor is 1:(0.1-2.0).

5. The preparation method for hexafluorobutadiene according claim 1, wherein the mesh size of the zinc powder is 100-500 meshes; the polar aprotic organic solvent used in the step (1) is selected from any one or more of tetrahydrofuran, N, N-dimethylformamide, N, N-dimethylacetamide, hexamethylphosphoramide, dimethyl sulfoxide, and N-methylpyrrolidone, and the moisture content of the polar aprotic organic solvent is ≤500 ppm, preferably ≤200 ppm.

6. The preparation method for hexafluorobutadiene according to claim 1, wherein the temperature range of the first reactor is 60° C.-120° C.; and the temperature range of the second reactor is 60° C.-90° C.

7. The preparation method for hexafluorobutadiene according to claim 1, wherein the settling device comprises a multi-stage settling tank.

8. The preparation method for hexafluorobutadiene according to claim 7, wherein the settling device includes two stages, namely a first settling tank and a second settling tank, the trifluoroethylene zinc bromide solution prepared in the step (1) is sequentially introduced into the first settling tank and the second settling tank through overflow or pumping, and the excess zinc powder is settled to obtain a trifluoroethylene zinc bromide solution without zinc powder residue.

9. The preparation method for hexafluorobutadiene according to claim 7, wherein the zinc powder in the settling tank in the step (1) can be directly used for the preparation reaction of the trifluoroethylene zinc bromide solution after pressure filtration.

10. The preparation method for hexafluorobutadiene according to claim 1, wherein the oxidant in the composite catalyst organic solution in the step (2) is selected from any one or more of sodium peroxide, potassium peroxide, sodium perborate, sodium persulfate, potassium persulfate, ammonium persulfate, and di-tert-butyl peroxide; the catalytic aid is selected from any one or more of cuprous chloride, cuprous bromide, cuprous iodide, ferrous chloride, and ferrous bromide; and the polar aprotic organic solvent used in the step (2) is selected from any one or more of tetrahydrofuran, N, N-dimethylformamide, N, N-dimethylacetamide, hexamethylphosphoramide, dimethyl sulfoxide, and N-methylpyrrolidone.

11. The preparation method for hexafluorobutadiene according to claim 1, wherein the molar ratio of trifluoroethylene zinc bromide to the oxidant in the step (2) is 1:(1.0-3.0); and the molar ratio of trifluoroethylene zinc bromide to the catalytic aid in the trifluoroethylene zinc bromide solution is 1:(0.01-0.2).

12. The preparation method for hexafluorobutadiene according to claim 1, wherein the temperature of the coupling reaction in the step (2) is −10° C.-50° C., the reaction pressure is 0 MPa-0.5 MPa, and the residence time in the reactor is 10 s-600 s; and the preferred temperature for the coupling reaction in the step (2) is 0° C.-10° C., the reaction pressure is 0 MPa-0.2 MPa, and the residence time in the reactor is 50 s-300 s.

13. The preparation method for hexafluorobutadiene according to claim 1, wherein in the step (3), the synthetic solution obtained in the step (2) is distilled to obtain a crude hexafluorobutadiene, and then rectified to obtain a pure hexafluorobutadiene.

14. The preparation method for hexafluorobutadiene according to claim 13, wherein the content of trifluoroethylene bromide in the crude hexafluorobutadiene obtained by distillation is ≤0.1%, the content of heptafluorobutene is ≤0.01%, and the purity of the product after distillation is ≥99.9%.

15. A preparation and production system for hexafluorobutadiene, wherein the preparation and production system is used for the preparation method according to claim 1, and included a trifluoroethylene zinc bromide solution preparation unit, a hexafluorobutadiene preparation unit, a zinc powder filtration unit, a distillation unit, and a rectification unit;
the trifluoroethylene zinc bromide solution preparation unit includes a trifluorobromoethylene organic solution feeding device, a zinc powder feeding device, a solvent and initiator feeding device, a first reactor, and a second reactor; wherein the trifluorobromoethylene organic solution feeding device, the zinc powder feeding device, and the solvent and initiator feeding device are connected to the first reactor for feeding; the zinc powder feeding device, and the solvent and initiator feeding device are connected to the second reactor for feeding; the first reactor is connected to the second reactor and is used to output the trifluoroethylene zinc bromide solution obtained from the reaction in the first reactor to the second reactor; the upper parts of the first reactor and the second reactor are connected to a vacuum and high-purity nitrogen device through a condenser, respectively; and an outlet of the second reactor is connected to a zinc powder settling device;
the hexafluorobutadiene preparation unit includes a trifluoroethylene zinc bromide solution feeding device, a composite catalyst solution feeding device, and a third reactor, and the trifluoroethylene zinc bromide solution feeding device and the composite catalyst solution feeding device are connected to an inlet end of the third reactor;
the distillation unit includes a hexafluorobutadiene synthetic solution feeding device, a distillation device, and a product collection device;
the rectification unit includes a crude hexafluorobutadiene feeding device, a rectification tower, a front distillate storage tank, a product collection tank, and a rectification residue storage tank.

16. The preparation and production system for hexafluorobutadiene according to claim 15, wherein the material of each equipment in the trifluoroethylene zinc bromide solution preparation unit and the hexafluorobutadiene preparation unit is selected from one of enamel glass, carbon steel, 316L, and fluorine resin lined carbon steel; the equipment material of the zinc powder filtration unit is selected from one of carbon steel, 304, and 316L; and the equipment material of the distillation unit is selected from one of enamel glass, 304, and 316L.

17. The preparation method for hexafluorobutadiene according to claim 4, wherein the ratio of the feed rate of the organic solution of the trifluorobromoethylene in kg/h to the mass value of the first reactor base material in kg is 1:(10-50); and/or the ratio of the feed rate value of the organic solution of the trifluorobromoethylene in kg/h to the mass value of the second reactor base material in kg is 1:(10-50).

18. The preparation method for hexafluorobutadiene according to claim 4, wherein the molar ratio of the trifluorobromoethylene to the feed rate of the zinc powder in the first reactor is 1:(1.0-3.0); and/or the molar ratio of the trifluorobromoethylene to the feed rate of the zinc powder in the second reactor is 1:(0.1-1.0).

19. The preparation method for hexafluorobutadiene according to claim 10, wherein the oxidant is selected from ammonium perborate, sodium persulfate, and potassium persulfate.

20. The preparation method for hexafluorobutadiene according to claim 10, wherein the catalytic aid is selected from any one or more of cuprous iodide and ferrous chloride.
